(19) United States
(12) Patent Application Publication
(10) Pub. No.: US 2012/0195984 A1
(43) Pub. Date: Aug. 2, 2012

(54) DIAGNOSIS AND TREATMENT OF THE PRODROMAL SCHIZOPHRENIC STATE

(76) Inventor: Jay L. Lombard, New City, NY (US)

(21) Appl. No.: 13/365,076

(22) Filed: Feb. 2, 2012

Related U.S. Application Data
(60) Provisional application No. 61/438,924, filed on Feb. 2, 2011.

(51) Int. Cl. A61K 33/00; G01N 33/573; G01N 33/566; A61K 31/65; A61B 5/055; A61K 31/202; A61K 31/198; C07C 53/126; A61P 25/18; C12Q 1/68; C07C 237/26

(52) U.S. Cl. 424/722; 435/6.12; 435/7.4; 436/501; 514/154; 552/205; 514/560; 514/562; 562/606; 600/410

(57) ABSTRACT

Described herein are compounds (including medical foods, pharmaceutical compositions, methods of compounding them), methods and systems for the diagnosis and/or treatment of prodromal schizophrenia. For example, described herein are methods of treating a developmentally-based neuropsychiatric disorder (schizophrenia) that includes first determining if a subject is at risk for such a disorder by examining phenotypical, serological immune markers and genotypical biomarkers. The biomarkers may be used to tailor the dose to be delivered by the medial food or pharmaceutical composition. Also described are compounds for treating prodromal (rather than full-blown) schizophrenia.

### CROSS REFERENCE TO RELATED APPLICATION

This patent application clams priority to U.S. Provisional Patent Application No. 61/438,924, filed on Feb. 2, 2011, and titled “TREATMENT OF THE PRODROMAL SCHIZOPHRENIC STATE”.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The compounds and methods described herein related generally to the treatment of neurodevelopmentally based disorders, and particularly the treatment of prodromal schizophrenia.

### BACKGROUND

Neuropsychiatric disorders such as schizophrenia are difficult to diagnose early and difficult to treat. However, there is a strong motivation to diagnose early, at preclinical or prodromal stages of the disorder, since early intervention may blunt, reduce or even prevent the full expression of this lifelong disease.

Because of current limitations in diagnosis, the prospective diagnosis of subjects as being in a prodromal risk syndrome for psychosis has yet to be accepted by psychiatric professional societies, the Food and Drug Administration, or US insurance companies. The absence of operational hallmarks of clinical validity has in turn slowed the development of a treatment research evidence base that could benefit these impaired, symptomatic, at-risk subjects and their families. One of the most important questions is whether the prodromal diagnosis can be refined so as to increase the proportion of cases that convert to psychotic illness.

Currently proposed “Risk Syndrome for Psychosis” (RS) criteria are derived from the Ultra High Risk criteria (UHR) and prodromal or Clinical High Risk criteria (CHR), and consist of subthreshold or attenuated positive psychotic symptoms. Identifying individuals meeting these criteria affords the possibility of early intervention to prevent or delay onset of full blown psychotic disorder. In particular, intervention in the prodromal phase of schizophrenia and related psychoses may result in attenuation, delay or even prevention of the onset of psychosis in some individuals. However, a “prodrome” is difficult to recognize prospectively because of its nonspecific symptoms.

Prodromic schizophrenia is often referred to as the onset phase of schizophrenia. Detection of prodromal schizophrenia would be beneficial because it could allow earlier and more specific treatment, which may ultimately prove more effective. However, detection and treatment of prodromal schizophrenia is difficult because there is no definitive test for prodromal schizophrenia, which is typically present before the subject starts actively hallucinating or exhibiting bizarre behavior characteristic of schizophrenia. The prodrome phase usually occurs one to two years before the onset of psychotic symptoms (for example: hallucinations, paranoid delusions) in schizophrenia. The symptoms people usually have during this time aren't very specific. Usually people report symptoms of anxiety, social isolation, difficulty making choices, and problems with concentration and attention. It is late in the prodromal phase that the positive symptoms of schizophrenia begin to emerge.

Three kinds of prodromal subgroups have been described: attenuated positive symptom syndrome; brief intermittent psychotic syndrome; and genetic risk plus functional deterioration. The attenuated positive symptom syndrome (APSS) classification is associated with problems with communication, perception, and unusual thoughts that don't rise to the level of psychosis. These symptoms have to occur at least once weekly for at least one month and become progressively worse over the course of a year. The brief intermittent psychotic syndrome (BIPS) prodrome subgroup is associated with problems with communication and perception, and the subject also experiences intermittent psychotic thoughts. The person experiences bizarre beliefs or hallucinations for a few minutes daily for at least one month, and for no more than three months. The last prodromal subgroup is the genetic risk plus functional deterioration group (G/D); these subjects are not currently psychotic but have been previously diagnosed with schizotypal personality disorder or they have a parent, sibling, or child that has been diagnosed with a psychotic disorder. Subjects are considered part of this subgroup if in the past year they have had substantial declines in work, school, relationships, or general functionality in daily life.

Although subject's may seek psychiatric help during the prodrome phase because of these disturbing symptoms, actual diagnosis of prodromal schizophrenia has proven extremely difficult, if not impossible, because these symptoms exist in many psychiatric and medical conditions. The problem of accurate diagnosis and treatment is particularly difficult for subjects who may be experiencing APSS or BIPSS prodromal schizophrenia. Many people experiencing prodromal schizophrenia that may later develop in to full-blown (late stage) schizophrenia) are misdiagnosed during the prodrome phase.

Although some highly significant predictors of psychosis have been found (e.g., long duration of prodromal symptoms, poor functioning at intake, low-grade psychotic symptoms, depression and disorganization) such behavioral predictors may be difficult to assess. The so-called SIPS criteria for a prodromal syndrome emphasize onset or worsening in the preceding 12 months of attenuated positive symptoms in one or more of five possible categories: unusual thought-content, suspicion/paranoia, perceptual anomalies, grandiosity, and disorganized communication. Such risk factors may be combined with other risk factors including family history, substance abuse, and ongoing mental state. Conclusively and rapidly determining such risk factors has proven difficult, however.

We herein propose rapid screens or tests to confirm prodromal schizophrenia that may be used to quickly and reliably determine that a subject is likely in a prodromal schizophrenic state. Such a test may evaluate a subject's genetic predisposition, including examining candidate genes involved in the pathogenesis of schizophrenia As discussed in greater detail below, the screening systems and methods described herein may confirm or suggest prodromal schizophrenia when a subject with a genetic susceptibility to schizophrenia experiences inflammation resulting in a decrease in the efficacy of the blood-brain barrier. We further disclose brain imaging modalities which may be incorporated for diagnosis of such prodromal states as well.

The majority of pathogenic genes are positively up-regulated, in part, via the action of the transcription factor NF-κB that plays key roles in orchestrating inflammatory responses and cell fate decisions. Other candidate genes in the pathogenesis of schizophrenia, involve interactions between neuregulin (NRG1) and glutamate receptors. The reported abnormalities in these receptor subtypes during brain development likely involve abnormal signaling related to axonal migration. The migrational defects in schizophrenia result in impaired cortical-subcortical-hippocampal communication networks. While the primary mechanisms involved in these migrational abnormalities are not completely understood, it is currently proposed that epigenetic and epistatic factors are critical to the emergence of these impairments. These epigenetic factors may be understood as representing a pathological cascade in which genetic abnormalities as disclosed increase vulnerability to the developmental regression features found in schizophrenia but require a “second hit” in order to reach clinical significance and clinical manifestations of overt symptomatology. The second hit relates to immunogenic factors and in many ways may be regarded as a brain specific autoimmune disorder. Several lines of evidence suggest that immunological factors contribute to schizophrenia. Increased activity, C3, C4 complement components, in schizophrenia has been reported as well as elevated plasma levels of sTNF-R1 and haptoglobin.

We herein propose that the prodromal (pre-symptomatic) diagnosis and treatment of psychosis or specifically schizophrenia may be established by both the concurrent presence of both a susceptibility to schizophrenia (e.g., as evidenced by a genetic polymorphism in one or more genes typically in a neurodevelopmental pathway implicated in schizophrenia) evidence of a pro-inflammatory condition that results in a decrease in blood-brain barrier efficacy, and objective neuroimaging parameters acquired through diffusion tensor imaging.

In addition, we herein describe therapies and treatments (including therapeutic compounds and compositions) that may be specifically used to treat prodromal schizophrenia, even in the absence of confirmation (e.g., by one of the tests or screens described herein). These compositions and treatments may include compositions known to increase the efficacy of the blood-brain barrier.

We further propose novel treatments directed at treating prodromal schizophrenia which may include: (1) detection of genetic vulnerability; (2) clinical confirmation by combining confirmatory serological biomarkers indicative of a pro-inflammatory state (particularly biomarkers indicating a decrease in blood-brain barrier efficacy), (3) obtaining diffusion tensor imaging to probe the integrity of brain white matter; and (4) basing treatment (even in the absence of full-fledged symptoms) on the concurrent presence of a psychosis succeptability marker, and/or a pro-inflammatory condition effecting the blood-brain barrier. Treatment even in the absence of full-fledged symptoms may therefore be based on treatment of the prodromal state alone, given a reliable indication of prodromal schizophrenia.

As mentioned briefly above, a primary need in the field of schizophrenia is the identification of etiologically significant biomarkers. Such identification, especially in preclinical or prodromal stages of these disorders, may provide a novel opportunity to reduce the probability of the full expression of these conditions, which if left untreated, almost invariably become chronic. It would clearly be desirable to identify a diagnostic tool for schizophrenia that is highly specific, and highly sensitive. However, in the absence of such a marker, the identification of factors associated with a higher probability of developing such a condition may be acceptable if the clinical response possesses a significantly lower risk to a child than a conventional medication. Of importance the marker should signify the disease early in its course, as there is evidence that delays in diagnosis and intervention lead to a poorer prognosis. In addition, a method that is cost-effective and non-invasive would be of added value. Given that subclinical or pre-clinical psychotic disorders may predict proneness, intervention in at risk individuals holds the promise of better outcomes. Thus, there is also a need for compositions, such as particularly medical food and pharmaceutical compositions, which are effective for treatment of psychosis such as schizophrenia in the prodromal state. It would be useful to provide such compositions to at-risk subjects, where risk is determined by one or more biomarkers indicating a susceptibility to such neuropsychiatric disorders. Described herein are candidate biomarkers and compositions (including medical foods and pharmaceutical compositions) that may be used to treat or prevent prodromal schizophrenia.

### SUMMARY OF THE DISCLOSURE

Described herein are systems, compositions, and methods of identifying and/or treating psychosis, and particularly prodromal schizophrenia. In particular, the methods, compositions and systems may be used to detect and/or treat prodromal schizophrenia. Thus, at least some of the systems described herein may therefore be referred to as prodromal schizophrenia detection systems or a prodromal schizophrenia treatment system; system may include diagnostics, kits, screens, therapies, and the like. At least some of the compositions described herein may be referred to as compositions for the treatment of schizophrenia (or prodromal schizophrenia); compositions may include medical foods, pharmaceuticals, and the like. The methods described herein may include methods of detection prodromal schizophrenia, methods of treatment of prodromal schizophrenia, and/or methods of detection and treatment of prodromal schizophrenia.

For example, described herein are systems, compositions, and methods for identifying individuals at risk for prodromal schizophrenia. A method or therapy for treatment of prodromal psychosis (such as schizophrenia) may include any of the following steps: identify a subject at risk for psychosis based on early symptoms (e.g., memory loss, changes in behavior, etc.); determine if the subject has a susceptibility to psychosis by family history and/or screening for the presence of genetic markers (e.g., single nucleotide polymorphisms) implicated as increasing the risk of susceptibility to psychosis; determine if the subject is in either (or both) a pro-inflammatory state and/or a state oxidative stress, and particularly a state indicative of weakening of the blood-brain barrier; indicating that the subject is in prodromal psychotic state if the subject is both genetically susceptible and experiencing a weakening of the blood-brain barrier (e.g., while in a pro-inflammatory and/or oxidative stress state); and treating the subject with a compound or composition (e.g., a compound containing N-acetyl cysteine). In some variations the step of determining the susceptibility and determining the pro-inflammatory state and/or oxidative stress (and/or the status of the blood-brain barrier) may be advantageously performed in the same assay.

The step of determining if a subject has a susceptibility to schizophrenia may include screening for genetic markers (e.g., polymorphisms) linked to schizophrenia in a pathway that is implicated in cell-signaling proteins that are involved in both brain development and in the inflammatory cascade. Examples of such genetic markers are described herein. The step of determining if the subject if a subject is experiencing a weakening of the blood-brain barrier may include determining if the subject is in either (or both) a pro-inflammatory state and/or a state oxidative stress, and may also be performed as part of the same screen as the genetic susceptibility mentioned above (e.g., using the same subject sample, or a concurrently taken sample), and may include examining the subject sample for pro-inflammatory markers or markers or oxidative stress, particularly those indicative of a dysfunction of the blood-brain barrier. Examples of these markers are described herein. In some variations, testing for pro-inflammatory markers or markers or oxidative stress may be a test for cerebritis.

For example, in one variation, a panel for determining if a subject is in a prodromal schizophrenic state (or at risk for being in such a state) may be a panel to determine levels of TNF, IL-1, IL-6, Haptoglobin, MMP-9 and S100B, or a sub-set of these.

A further step in confirmation comprises measuring the integrity of white matter via utilization of diffusion tensor imaging. Limitations of current diffusion tensor imaging are discussed and methods on how to overcome these limitations in clinical settings are disclosed

Also described herein are reports that may accompany results of any of the screens described herein, which may inform the physician of test results as well as providing an interpretive guide, e.g., indicating the likelihood of prodromal schizophrenia based on one or more additional factors.

Thus, the methods and compositions described herein may relate in general to a method of identifying phenotypical and genotypical biomarkers in preclinical or prodromal stages of a pediatric neuropsychiatric disorder and subsequently addressing the risk by potentially inhibiting the clinical expression of said disorder through the employment of a safe medical food compound.

As mentioned, also described herein are compounds for the treatment of prodromal schizophrenic states. In some variations the compounds may be compositions including low dose lithium, essential fatty acids and an acetylcysteine compound (e.g., NAC). This composition or compound may be particularly advantageous for the treatment of prodromal schizophrenia. In some variations of the compounds for treatment of prodromal schizophrenia are compounds that enhance the blood-brain barrier and/or compounds that prevent damage to the blood-brain barrier. For example, described herein are compounds including one or more inhibitor of MMP-9 to treat prodromal schizophrenia.

The fatty acid compounds described herein that may be part of the compositions or compounds include essential fatty acids (EFAs), such as alpha-linolenic acid (an omega-3 fatty acid) and linoleic acid (an omega-6 fatty acid). Other fatty acids that may be used include conditionally essential fatty acids, such as gamma-linolenic acid (an omega-6 fatty acid), lauric acid (a saturated fatty acid), and palmitoleic acid (a monounsaturated fatty acid).

This invention also provides a method for preventing or treating prodromal schizophrenia in a subject comprising administering to the subject a composition such as those described herein.

### BRIEF DESCRIPTION OF THE DRAWINGS

FIG. 1A illustrates a model for entering prodromal schizophrenia.

FIG. 1B schematically illustrates a method of diagnosing prodromal schizophrenia as described herein.

FIG. 2A schematically illustrates one method of determining prodromal risk for schizophrenia.

FIG. 2B schematically illustrates one method of determining prodromal risk for schizophrenia.

### DETAILED DESCRIPTION

In general, the systems, compositions and methods described herein may be used to diagnose and/or treat prodromal psychosis. Although the examples described herein are specific to the diagnosis and treatment of schizophrenia, other psychosis may be similarly diagnosed and/or treated.

In general, methods of determining if a subject is at risk for developing schizophrenia may include (1) confirming that the subject is experiencing symptoms consisted with prodromal schizophrenia; (2) determining if the subject is susceptible to developing schizophrenia based on a genetic susceptibility (“genetic susceptibility for schizophrenia”); and (3) determining if the subject is under either (or both) oxidative stress or a pro-inflammatory state (“current oxidative/inflammation stress state”) with a decrease in blood-brain barrier (BBB) function and (4) applying novel diffusion tensor imaging modalities to confirm diagnosis. Thus, described herein are systems, such as screens or tests, which may determine at least some of these factors, as well as reports for reporting and providing interpretive aid to the results of these screens helpful for accurately and rapidly diagnosing prodromal schizophrenia. The methods, systems and/or reports may also help guide therapeutic decisions for treating a subject. The reports described herein may include reports describing the prodromal status of the subject by aggregating and indicating the susceptibility for schizophrenia and the status of the subject's blood brain barrier and/or inflammatory (and/or oxidative stress) state.

Compounds for treating a subject experiencing or at risk for prodromal schizophrenia are also described in greater detail below. In some variations, the compounds or compositions include one or more agents for improving the function of the blood-brain barrier or preventing damage to the blood-brain barrier. For example, in some variations the compounds or compositions include one or more inhibitors of MMP-9, such as minocycline. In some variations the compounds or compositions generally include a formulation compound and an acetylcysteine compound, combined with low dose lithium and essential fatty acids.

As mentioned, in some variations, these compounds are prescribed if a test/screen/panel for prodromal schizophrenia (as described herein or elsewhere) indicates a high likelihood of prodromal schizophrenia. Alternatively, these compounds, and particularly the medical food variations, may be prescribed or taken even in the absence of a confirmation of prodromal schizophrenia. These compounds may also be used to treat existing (as opposed to prodromal) schizophrenia. The compounds and methods described herein may be configured as medical foods. The formulation of these compounds, as well as the application or use of these compounds as medical foods to treat subjects in need thereof is described in greater detail below, including by example.

As used herein, a subject may be a patient, and may be any subject, including humans.

As used herein the phrase “medical food” may refer to foods that are formulated and intended for the dietary management of a disease or disorder. These foods may provide distinctive nutritional elements that cannot be met by normal diet alone. Medical foods may be distinct from the broader category of foods for special dietary use and from traditional foods that bear a health claim. A medical food may be a food for oral ingestion or tube feeding (nasogastric tube), may be labeled for the dietary management of a specific medical disorder, disease or condition for which there are distinctive nutritional requirements, and may be intended to be used under medical supervision. Examples of medical foods may include: nutritionally complete formulas, nutritionally incomplete formulas, and formulas for metabolic disorders. Although the variations and examples described herein are specific to medical foods, in some variations the compositions described herein may be prepared and/or compounded as traditional “drugs” or medicaments.

### Part I: Assessing Prodromal Schizophrenia

In general, a prodromal schizophrenic state may be assessed by examining (1) subject behavioral characteristics associated with prodromal schizophrenia; (2) genetic susceptibility to schizophrenia; and (3) a weakening of the blood-brain barrier. In some variations the etiology of the prodromal state may be examined by looking at both or either the particular genetic factors providing susceptibility to prodromal schizophrenia and the trigger state (e.g., the inflammatory state and/or oxidative stress) which correlates with the prodromal schizophrenia. If a patient is exhibiting characteristics consistent with prodromal schizophrenia, and has a genetic susceptibility to schizophrenia and is experiencing a decrease in blood-brain barrier function, the subject is likely to be experiencing prodromal schizophrenia, and appropriate treatment may be indicated. The treatment indicated may be further refined based on the degree of behavioral characteristics, the type, class or extent of genetic susceptibility, and the etiology and/or extent of weakening of the blood-brain barrier.

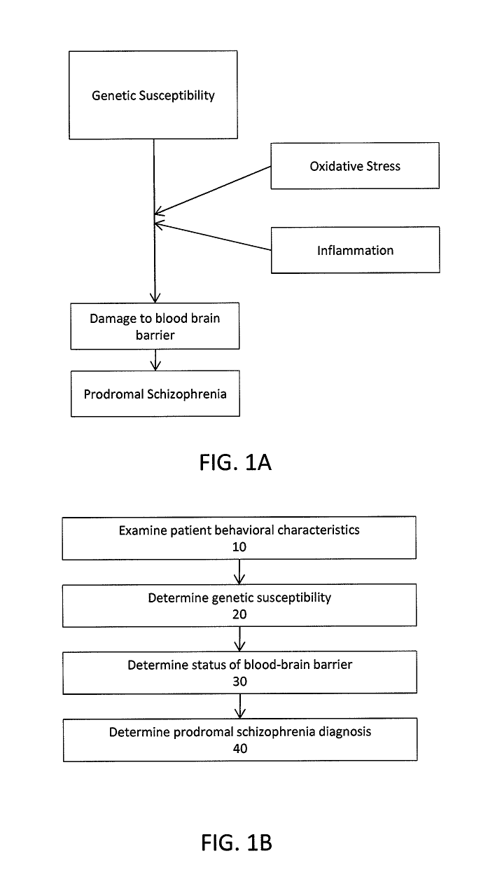

Without being bound to a particular theory, the inventor has hypothesized that prodromal schizophrenia may be triggered in a particular individual when the individual has a genetic susceptibility for schizophrenia and is “triggered” to enter the prodromal schizophrenic state by an inflammatory response and/or an oxidative stress. This is illustrated diagrammatically in FIG. 1A. In this example, genetic susceptibility for schizophrenia may be a result of certain mutations or dysfunctions of genes, typically genes that are implicated both in neurodevelopment and the immune response, such as neuregulin. As illustrated in FIG. 1A, when the subject having a genetic susceptibility for schizophrenia is triggered by an inflammatory response (e.g., potentially due to infection, injury, or any other cause) and/or oxidative stress, the result is a weakening of the blood-brain barrier, which may result in decrease in the integrity of the blood-brain barrier (e.g., partial breakdown). This decrease in function of the blood-brain barrier in tern leads to prodromal schizophrenia, and may ultimately progress into full-blown schizophrenia. Importantly, under this model even prodromal schizophrenia (which is otherwise difficult to diagnose and treat) may be detected with confidence, in subjects (1) having behaviors consistent prodromal schizophrenia where the subject (2) has a genetic susceptibility to schizophrenia and (3) has a weakened blood-brain barrier, and in some variation, (4) acquiring tensor imaging to interrogate the integrity of white matter in the brain Thus, if all or most of these factors are present, the subject may be diagnosed and treated for prodromal schizophrenia.

Further, under this model the weakening of the blood-brain barrier is believed to be triggered in subjects with a genetic susceptibility when a trigger stimulus (e.g., inflammation, oxidative stress, etc.) is present. Thus, detection of inflammation and/or oxidative stress in the subject (in addition to weakening of the blood-brain barrier) may determine the etiology of the prodromal state, which may further refine the proposed treatments. Alternatively, in some variations, detection of the presence of inflammation and/or oxidative stress in the subject may be used as a proxy for a determination of prodromal schizophrenia, when factors (1) and (2) are also present (e.g., the non-specific symptoms consistent with prodromal schizophrenia and a genetic predisposition to schizophrenia). FIG. 1B outlines one application of the method of diagnosing prodromal schizophrenia on the basis of the model described above. This model (and theory) is elaborated in greater detail below. It should be apparent to those of skill in the art that the methods, systems and compounds/compositions for treating and diagnosing prodromal schizophrenia described herein are not dependent on this model and theory, and may function as claimed even in the event that the theoretical framework is incomplete or even incorrect. Thus, the inventor does not wish to be bound by the model and theory described herein.

The pro-inflammatory state associated with prodromal schizophrenic states has been observed in a research context but has been previously unrealized clinically. Despite theoretical causal associations, the ability to diagnosis prodromal schizophrenia states clinically, based upon recognition of biomarkers indicative of such a state, as well as the implementation of specific anti-inflammatory agents prescribed to inhibit the adverse effects of such, have not yet been realized. To the inventor's knowledge, the pathological link between a pro-inflammatory state in prodromal schizophrenia and alterations in the blood brain bather has not been previously explored as either a diagnostic or therapeutic target for prodromal schizophrenia.

The blood-brain barrier is a semi-permeable membrane composed of endothelial cells connected by tight junctions. It functions as a physiological barrier which dynamically regulates the exchange of substances from the vascular system and brain. Increased permeability of the blood brain barrier has been demonstrated in a variety of neurological disorders including Alzheimer's, multiple sclerosis and stroke but is less recognized as an association with schizophrenia or other psychiatric states. The mechanisms related to blood brain barrier alterations are varied but are linked to pro-inflammatory states and the expression of cytokines and matrix metallic proteinases which increase endothelial cell permeability.

Thus, the ability to identify abnormal changes in blood brain-barrier permeability, primarily as a means to validate prodromal schizophrenic states, but also as a means to identify other abnormal psychiatric states associated with such changes, is critically needed as an improvement in the field of clinical neuropsychiatric diagnosis.

Current methods to determine BBB breakdown are limited by cost (contrast-enhanced MRI) or invasiveness (lumbar puncture). Neither is suitable for broad-scale or frequent screening of populations at risk. Thus, a surrogate marker of BBB function offers several advantages. Various techniques to identify blood-brain barrier alterations are thus herein described which can be applied specifically as an aide in confirming the diagnosis of a pro-inflammatory state associated with prodromal psychotic states such as schizophrenia. In some variations, the method includes the use of an assay which includes biomarkers related to pro-inflammatory cytokines, matrix metalloproteinaces, which may specifically include MMP-9 and S100B.

S100B is believed to be a prevalent protein of the central nervous system, and may be used as a peripheral biomarker for blood-brain barrier disruption and often also a marker of brain injury. Studies have shown that subjects suffering from depression or schizophrenia may have immunological alterations that can be detected in the blood. Others reported a possible link between inflammation, a microgliosis and the blood-brain barrier (BBB) in suicidal subjects. Serum S100B may be used as a marker of BBB function, and elevated levels of S100B may be regarded as one indication of blood brain barrier disruption.

The MMPs are believed to have influence in several normal processes, and the physiological and preanalytical factors affecting these enzyme levels should be carefully identified in order to accurately use these enzymes as psychiatric biomarkers; sample type has been found to have an effect on the concentrations of metalloproteinases in circulating blood. For example, sample type has been shown to have the clearest effect on the levels of pro-MMP-9. Platelets contain both MMP-9 and TIMP-1, and it has been shown that platelet aggregation during clotting can lead to increased release of MMP-9. It has also been shown in several studies that serum has generally higher levels of MMP-9 than do plasma samples. This has been documented using both ELISA and gelatin zymography. For instance, coagulation activators had an effect on the pro-MMP-9 serum levels, giving up to 4-fold MMP-9 levels in comparison with native serum, probably due to platelet release of MMP-9. Since pro-MMP-9 is sensitive to several preanalytical issues (e.g., coagulation activators, anticoagulants, storage time), standardization is crucial if this enzyme is to be measured from circulation. For example, MMP-9 levels are affected by coagulation activation and the anticoagulant used, and MMP-9 may therefore be more safely determinable in plasma samples.

Returning to FIG. 1B, a schematic example of an overall method of determining the risk of prodromal schizophrenia is illustrated. In the first step illustrated in FIG. 1B, the subject is assessed for behaviors characteristic for prodromal schizophrenia 10. For example, behavioral characteristics of prodromal schizophrenia may include neurotic symptoms, mood-related symptoms, changes in volition, cognitive changes, physical changes, behavioral changes and additional symptoms.

For example, neurotic symptoms typically include: anxiety, restlessness, anger, and irritability. Mood-related symptoms typically include: depression, anhedonia, guilt, suicidal ideas, mood swings. Changes in volition typically include: apathy (loss of drive), boredom (loss of interest), and fatigue (loss of energy). Cognitive changes typically include: disturbance of attention, inability to concentrate, preoccupation (daydreaming), thought blocking, and reduced abstraction. Physical symptoms typically include: somatic complaints, loss of weight, poor appetite, and sleep disturbance. Behavioral changes typically include: deterioration in school, work, or other role functioning; social withdrawal; impulsivity; odd behavior; and aggressive (disruptive) behavior. Additional symptoms may include: obsessive compulsive phenomena; dissociative phenomena; increased interpersonal sensitivity; change in sense of self, others, or the world; change in motility; speech abnormalities; perceptual abnormalities; suspiciousness; and change in affect.

The initial prodromal symptoms in schizophrenia were studied in 100 DSM-diagnosed subjects and 100 controls. The median number of symptoms in the subjects and the controls was 8 (range 2-13) and 0 (range 0-5), respectively. Subjects developed symptoms indicating social, occupational, and affective dysfunction, whereas the controls' symptoms included magical content and disturbance in mood. There were significant differences in the frequency of several symptoms appearing in the subtypes. Initial prodromal symptoms were classified into negative, positive-prepsychotic, and positive-disorganization categories. Subjects with the disorganized subtype were more likely to have had negative symptoms in the prodromal state, and subjects with the paranoid subtype were more likely to have had positive symptoms in the prodromal state. Observation of the course of symptoms from the prodromal to the psychotic state revealed that 58 percent of the symptoms showed increased intensity, 21 percent remained unchanged, 5 percent decreased, 3 percent evolved into other affective difficulties, 9 percent progressed into delusions, 1 percent progressed into hallucinations, and 3 percent disappeared. The Global Assessment of Functioning Scale showed that functioning is differentially affected among the subtypes even in the prodromal phase. These findings provide a better understanding of the initial prodromal state of schizophrenia, the signs and symptoms that best define it, and their prognostic significance.

If the subject is positive for any of the behavior characteristics consistent prodromal schizophrenia, such as those described above, the subject may be prodromal (e.g., prodromal schizophrenic), however these characteristics alone are not sufficient to make a determination, as they are not specific to prodromal schizophrenia. As illustrated in FIG. 1B, a method of diagnosing or analyzing prodromal schizophrenia may also include the steps of determining if the subject also has a genetic susceptibility to schizophrenia, and determining the likelihood that the blood-brain barrier has been weakened.

In general, the step of determining a genetic susceptibility to schizophrenia may include either or both a genetic screen of the subject, looking at genes or genetic regions implicated in susceptibility for schizophrenia (described in reference to Table 1 in greater detail below), and/or examining the subject's own and family psychiatric history. For example, an examination of the subjects own and family psychiatric history may if the subject has been previously diagnosed with schizotypal personality disorder, or they have a parent, sibling, or child that has been diagnosed with a psychotic disorder; if so, the subject may have a genetic susceptibility for schizophrenia. Since family and personal psychiatric history may be difficult to determine and may not be fully representative, at least some genetic screening to determine or confirm genetic susceptibility may also be advised. As with the non-specific behavioral characteristics, evidence of a genetic predisposition (even in conjunction with the presence of behavioral characteristics) is typically not sufficient to determine prodromal schizophrenia. Thus, evidence that the blood-brain barrier has been weakened (and/or evidence of inflammation/oxidative stress) may also be necessary to conclusively determine prodromal schizophrenia.

Although the pathophysiology of schizophrenia remains unclear, there is an increasing body of evidence that several molecular pathways are involved. Neuroanatomical changes observed in psychotic disorders of childhood suggest an active biological process during the transition to full blown disease expression, raising the possibility that intervention might be indicated prior to expression of frank psychotic symptoms.

Genes may include NRG1 and many of its downstream signaling components (e.g., AKT1, etc.). For example, the NRG1α-induced adhesion response is dependent on signaling through Akt pathways. Perturbations in neuregulin-1 (NRG1)/ErbB4 function have been associated with schizophrenia. Affected subjects exhibit altered levels of these proteins and display hypofunction of glutamatergic synapses as well as altered neuronal circuitry. ErbB4-mediated synapse maturation requires its extracellular domain, whereas its tyrosine kinase activity is dispensable for this process. Depletion of ErbB4 decreases the number of primary neurites and stimulation of ErbB4 results in exuberant dendritic arborization through activation of the tyrosine kinase domain of ErbB4 and the phosphoinositide 3-kinase pathway.

Nitration of NRG-Fs (nNRG-1) EGF-like domain results in an inability to activate its receptor, Thus nitration of NRG-1's EGF-like domain caused it to lose its ability to bind and activate its receptor with loss of ligand-induced proliferation. The therapeutic effect of inhibiting tyrosine nitration via the nitration inhibitor/thiol donor N-acetylcysteine may restore Akt phosphorylation and subsequently restore normal NRG development signals.

Recent evidence suggests that NRG1 may play a role in regulation of inflammation and immune system response. Schizophrenia-associated miss-sense mutations within the transmembrane region of NRG1 may also be linked to immune dysregulation. In vivo, increased levels of 25 autoimmune markers as well as elevated levels of cytokines were significantly associated with the NRG1 mutation. Increase in protein secretion levels of IL-6, TNF-α, and IL-8 were also present in NRG mutation carriers compared with controls.

Glutathione (GSH) is the major free radical scavenger in the brain. Diminished GSH levels elevate cellular vulnerability towards oxidative stress; characterized by accumulating reactive oxygen species. Levels of reduced, oxidized, and total GSH were significantly decreased. Consistent with the disclosure herein, accruing data suggest that oxidative stress may be a critical factor underlying the pathophysiology of schizophrenia. Post-mortem prefrontal cortex from subjects with each of these disorders have found that the levels of reduced, oxidized, and total Glutathione (GSH) were significantly decreased in all psychiatric conditions compared to the control groups. Results suggested an enhanced generation of reactive oxygen species and significantly lower free radical scavenging capacity in schizophrenia subjects compared to healthy controls.

Indicators of oxidative stress are detectable in the urine and blood of many schizophrenic subjects. Significantly increased levels of isoprostanes were observed among schizophrenia subjects relative to the controls, as measured by isoprostane-8-epi-prostaglandin F(2alpha) (8-isoPGF(2alpha)) concentrations in the urine. In further support that vulnerability to schizophrenia may be mediated by diminished brain antioxidant systems, microarray studies demonstrate up-regulation of SELENBP1 (selenium binding protein) in the brain and blood of subjects with schizophrenia. Results demonstrate that SELENBP1 mRNA is unregulated in schizophrenic brains versus controls and, in addition, that SELENBP1 gene expression is strongly positively correlated with presence of psychosis across diagnoses. Furthermore, organic selenium compounds have been demonstrated to significantly reduce apomorphine-induced stereotyped behaviors in animals.

These lines of evidence point to the utility of raising antioxidant brain defense systems to mitigate the risk of developing a childhood psychotic disorder such as schizophrenia. In particular, glutathione activity may be neuroprotective in these disorders by its influence on receptor interactions within receptor heterodimers and receptor mosaics, representing an important integrative mechanism for signaling based upon redox sensitive mechanisms in brain networks.

Modulation of glutamatergic transmission through distinct and selective receptor subtype mechanisms, such as potentiation of the N-methyl-D-aspartate (NMDA) receptor glycine site, activation of group II mGluR, and activation of glutamate-cystesine antiporters represent novel neurochemical targets to treat schizophrenia. Thus, the potential ability to positively modulate these receptors via the augmentation of brain glutathione by administration of a specific medical food represents a novel treatment.

The tripeptide glutathione (gamma-glutamylcysteinylglycine) is the primary endogenous free radical scavenger in the brain. When glutathione (GSH) levels are reduced there is increased cellular oxidative stress, characterized by an increase and accruement of reactive oxygen species (ROS). This may result in alterations in dopaminergic and glutamatergic activity implicated in these illnesses. Glutamate and dopamine are highly redox reactive molecules and produce free radicals during neurotransmission. Neurons are thus at high risk for oxidative injury and pro oxidative states have detrimental consequences on normal migrational processes and brain connectivity during development.

Synthesis of glutathione, a major redox regulator, is compromised in schizophrenia. The glutathione deficit, via its effect on redox-sensitive proteins could contribute to dysfunction of neurotransmitter systems in schizophrenia. Experimental models of glutathione deficit changed the modulation of responses by dopamine, from enhanced responses in control neurons (likely via D1-type receptors) to decreased responses in low-glutathione neurons (via D2-type receptors). This difference in dopamine modulation was due to a different modulation of L-type calcium channels activated during NMDA stimulation: dopamine enhanced function of these channels in control neurons but decreased it in low-glutathione neurons. The effect of a glutathione deficit on dopamine signaling was dependent on the redox-sensitive ryanodine receptors (RyRs), whose function was enhanced in low-glutathione neurons. This suggests that enhanced RyRs in low-glutathione neurons strengthens intracellular calcium-dependent pathways following activation of D2-type receptors and causes a decrease in function of L-type channels. This represents a mechanism by which dopaminergic systems could be dysfunctional under conditions of impaired glutathione synthesis as in schizophrenia. These changes closely mimic the pathological imbalances of dopamine signaling in schizophrenia, where D1 receptor function is blunted and D2 receptor activity is exaggerated.

Wistar rats treated with phencyclidine (10 mg/kg) exhibit region-specific changes characterized by decreased content of reduced glutathione (GSH). In hippocampus, reduced GSH content and decreased activities of GPx are induced by PCP administration. Furthermore, GSH-deficient mice displayed an increased locomotor response to low (2 and 3 mg/kg, i.p.) doses of phencyclidine. Moreover, the open field findings suggest reduced or altered N-methyl-d-aspartate (NMDA) receptor function in GSH-deficient mice.

Genetic studies have shown an association between schizophrenia and a GAG trinucleotide repeat (TNR) polymorphism in the catalytic subunit (GCLC) of the glutamate cysteine ligase (GCL), the key enzyme for glutathione (GSH) synthesis. This altered pattern potentially contributes to the development of a biomarker profile useful for early diagnosis and monitoring the effectiveness of novel treatments targeting redox dysregulation in schizophrenia.

N-acetyl cysteine (NAC) is a precursor of cysteine and glutathione. It has antioxidant properties, lipid stabilization, and preservation of mitochondrial membrane potential, all of which may favorably impact receptor function in neuropsychiatric states. Treatment of neurons with lipid peroxidation byproducts results in a drastic reduction of mitochondrial membrane potential, and this reduction is prevented by NAC. This neuroprotective effect is due, at least in part, to preservation of mitochondrial membrane potential and intracellular GSH levels. Thus, NAC may exert neuroprotective effects via its ability to inhibit oxidation of mitochondrial proteins, and stabilization of receptor membrane dimers. Other variations or forms of NAC may be used; for example, N-acetylcysteine amide (NACA).

NAC is also a potent glutamate modulator in the brain via its effects on the glutamate/cystine antiporter. The glutamate/cystine antiporter x(c)- transports cystine into cells in exchange for glutamate at a ratio of 1:1. Glutamate exported by system x(c)- is largely responsible for the extracellular glutamate concentration in the brain, whereas the imported cystine is required for the synthesis of the major endogenous antioxidant, glutathione. System x(c)- thus connects the antioxidant defense with neurotransmission and behavior. Disturbances in the function of system x(c)- have been implicated in nerve cell death due to increased extracellular glutamate and reduced intracellular glutathione. In vitro, inhibition of cystine import through system x(c)- leads to cell death by a mechanism called oxidative glutamate toxicity, which includes depletion of intracellular glutathione, activation of 12-lipoxygenase, accumulation of intracellular peroxides, and the activation of a cyclic guanosine monophosphate (cGMP)-dependent calcium channel towards the end of the death cascade. N-acetyl cysteine (NAC) inhibits glutamate via the cystine-glutamate exchange system. Further, by boosting glutathione, NAC acts as a potent antioxidant and has been shown in two positive, large-scale randomized placebo-controlled trials to affect negative symptoms in schizophrenia and depression in bipolar disorder.

N-acetylcysteine (NAC) treatment may exert its effects by activating cystine-glutamate exchange and thereby stimulating extrasynaptic metabotropic glutamate receptors (mGluR). NAC treatment of rats restored the ability to induce formation of new memories by indirectly stimulating mGluR2/3 and mGluR5, respectively. Thus, a previously undisclosed mechanism whereby NAC exerts beneficial effects in cognitive decline in pediatric neuropsychiatric disorders involves the facilitation of glutamate efflux and reduction of glutamate mediated excitotoxicity. N-acetyl cysteine (NAC) as an add-on to maintenance medication for the treatment of chronic schizophrenia has potential as a safe and moderately effective augmentation strategy for chronic schizophrenia. While the use of NAC has been proposed to be employed in clinical states of schizophrenia, its application and use in prodromal states and for the explicit purpose of preventing schizophrenia has not been previously disclosed (see, e.g., H H Chen, A Stoker, and A Markou, Psychopharmacology (Berl), 2010 May; 209(4):343-50).

The proposed mechanism linking oxidative stress with membrane lipid abnormalities, inflammation, aberrant immune response, impaired energy metabolism and excitotoxicity, leading to clinical symptoms and pathogenesis of schizophrenia, suggests that interventions which restore anti oxidant defense systems may reduce the vulnerability to the expression of this disorder. Thus, described herein are methods of treating prodromal (e.g., pre-clinical) schizophrenia with NAC and particularly compositions containing NAC.

Low dose lithium: low-dose lithium (1 mg/kg) may counteract the microstructural and metabolic brain changes in individuals with prodromal states. Low-dose lithium significantly protects the microstructure of the hippocampus in as reflected by significantly decreasing hippocampal T2 relaxation times.

700 mg of eicosapentaenoic acid (20:5n3), 480 mg of docosahexaenoic acid (22:6n3) has been demonstrated to reduce the risk of progression to psychotic disorder and may offer a safe and efficacious strategy for indicated prevention in young people with sub-threshold psychotic states or prodromal states

While each of these components; NAC, low dose lithium, and essential fatty acids have been theoretically applied separately to reduce the risk of progression from prodromal states to schizophrenia or to ameliorate schizophrenic symptoms, the combination of these components has not previously been disclosed

### Additional Compounds for Treatment of Prodromal Schizophrenia

In general, a compound or composition for treatment of prodromal schizophrenia may modulate the blood-brain barrier, and/or may inhibit elements that weaken the blood-brain barrier. For example, in some variations a composition or compound for treatment of prodromal schizophrenia includes an inhibitor of MMP-9. MMP-9 inhibitors may therefore reduce blood-brain barrier permeability and therefore be of potential benefit in the treatment of prodromal schizophrenia may include, for example: doxycycline and minocycline, valproic acid and other HDAC inhibitors, lithium, NAC and Fish oils have been demonstrated to improve the integrity of the blood brain barrier

For example, VPA (Valproic acid) significantly reduces elevation of matrix metalloproteinase-9 (MMP-9), and prevents degradation of tight junction proteins, and nuclear translocation of nuclear factor-κB (NF-κB) and may be a neuroprotective agent

The amount of MMP-9 inhibitor to be used is typically sufficient to reduce or inhibit MMP-9 activity or expression to restore or allow the blood-brain barrier to be restored to normal permeability. Thus, in some variations this amount may be determined based on clinical evidence or trials examining the effect of the MMP-9 inhibitor on the blood-brain barrier or directly on the characteristics behaviors associated with prodromal schizophrenia.

An important element of the discovery relates to the critical importance of maintaining adequate blood levels of the medical food or drug composition. NAC, Lithium, essential fatty acids, minocycline and Valproic acid all typically has a short half life; delayed-release (“slow-release”) forms may therefore be used. After an oral dose of N-acetylcysteine 200 to 400 mg has a terminal half-life of 6.25 h. Thus, to achieve a therapeutic response in schizophrenia, an individual may require frequent dosing. Therefore, an improvement in the application of these compounds may involve a controlled delivery mechanism that would ensure continuous blood levels to achieve a desired therapeutic response.

In some variations, methods for treating the subject may include first determining if the subject is at risk for or is currently suffering from prodromal schizophrenia.

### Methods of Identifying Prodromal Schizophrenia

The methods described herein may include determining genetic susceptibility for schizophrenia, determining if the blood-brain barrier is weakened or damaged, and in some variations, determining if the subject is under inflammation and/or oxidative stress (which may have led to the weakening of the blood-brain barrier). Correlation of genetic susceptibility with one or more direct measures of blood-brain barrier status (and/or peripheral immune system and/or oxidative stress) may allow diagnosis of prodromal schizophrenia at a level of certainty which will mandate a treatment intervention in subjects expressing behaviors consisted with prodromal schizophrenia.

Thus, in general the systems described herein may include a screening panel incorporating both: (1) one or more indicators of genetic susceptibility for schizophrenia; and (2) and one or more indicators of blood-brain barrier function; in some variations the panel may also include one or more indicators of an ongoing pro-inflammatory state (e.g., inflammation such as cerebritis) or one or more indicators of oxidative stress. (3) Obtaining diffusion tensor imaging for confirmational diagnosis

An indicator of genetic susceptibility for schizophrenia may include an indicator evidencing the presence of a genetic marker linked to an elevated risk of schizophrenia. In particular, the marker may be for one or more genes related to brain development signals (such as neuregulin), which also may function in neuro-immune based pathways. Example include ZNF804, MHC, DISCI, AKT, and specifically those variations of these genes (e.g., Single Nucleotide Polymorphisms or SNPS) suggesting heightened genetic vulnerability. Table 1, below illustrates some of these genes.

For example, genetic markers for susceptibility to schizophrenia may include gene polymorphisms in modulatory systems involving the glutamate receptor (NMDAR), and enzymes of the oxidative pathways related to glutathione and neuregulin. Thus, these genes may be examined to determine the genetic susceptibility for schizophrenia; certain forms, including certain polymorphisms, of these genes may be indicative of positive or enhanced genetic susceptibility for schizophrenia. For example, Altered neuregulin (NRG1) in brain development may be relevant to the pathophysiology of schizophrenia and dysfunction of the NMDA receptor. NRG1 normally acts to promote NMDA activity via the phosphorylation of the NR2B subunit. Abnormal NRG1 signaling reduces NR2B and subsequently impairs NMDA receptor function. Other genes (and polymorphisms) are also described below for possible inclusion in determining genetic susceptibility for schizophrenia.

In general, the markers that may be tested to determine genetic susceptibility for schizophrenia are not limited to markers of genetic polymorphisms. In general, any appropriate biomarker may be used. Biomarkers may be in the form of genes, proteins and other molecules, or phenotypical characteristics. Depending on the information they can provide, biomarkers may be used in diagnostics as prediction tools (e.g. subclinical markers, risk or vulnerability markers), or as diseases signatures (e.g. disease markers, stage or progression markers). An endophenotype may be neurophysiological, biochemical, endocrinological, neuroanatomical, cognitive, neuropsychological or genetic.

TABLE 1
Genetic variants indicating an increased
susceptibility to schizophrenia

| Gene | Variation(s) | Comment |
|---|---|---|
| NRG1 | rs3924999 | G to A base |
| (Neuregulin 1) | | change in position |
| | | 12 of the second |
| | | exon of NRG1 |
| | | (neuregulin 1) |
| | rs2954041 | |
| | SNP8NRG221533 | |
| | SNP8NRG241930 | |
| | SNP8NRG243177 | NRG1 promoter |
| | rs1081062 | located in intron 1 |
| | | of NRG1 |
| NRG3 | rs10883866 | intron 1 of NRG3 |
| (Neuregulin 3) | rs10748842 | intron 1 of NRG3 |
| | rs6584400 | intron 1 of NRG3 |
| ZNF804 | rs1344706 | |
| (Zinc Finger Protein 804A gene) | | |
| MHC | rs3130375 | affects the RPP21 |
| (Major Histocompatibility | | gene (a subunit of |
| Complex) | | nuclear |
| | | ribonuclease P) |
| | rs13194053 | within a histone |
| | | gene cluster |
| | rs3131296 | within the |
| | | NOTCH4 locus |
| DTNBP1 | rs9370822 | |
| (Dysbindin or dystrobrevin | | |
| binding protein) | | |
| DISC1 | rs3738401 | |
| (Disrupted in Schizoprenia-1) | rs6675281 | |
| PIP5K2A | rs10828317 | |
| RGS4 | rs10917670 | |
| (RGS4-1 or | | |
| AKT1 | rs1130233 | |
| (A protein-serine/threonine | | |
| kinase 1) | | |
| ACSL6 | rs11743803 | |
| (Acyl-Coenzyme A synthetase | | |
| long-chain family member 6) | | |
| COMT | rs165599 | |
| (CATECHOL-O- | rs4680 | (Val158Met) |
| METHYLTRANSFERASE) | | |
| SYNII | rs310762 | |
| (Synapsin II) | rs795009 | |
| ERBB4 | rs707284 | |
| (V-ERB-B2 AVIAN | rs7598440 | |
| ERYTHROBLASTIC | rs839523 | |
| LEUKEMIA VIRAL | | |
| ONCOGENE HOMOLOG 4 | | |
| DAOA | rs947267 | |
| (D-amino acid oxidase activator) | | |
| MEGF10 | rs27388 | |
| (multiple EGF-like-domains 10) | | |
| SLC18A1 | rs2270641 | |
| (VMAT1, encodes the vesicular | | |
| monoamine transporter 1) | | |
| FGFR2 | rs17101921 | maps 85 kb from |
| (fibroblast growth factor | | the nearest gene |
| receptor 2) | | encoding |
| | | fibroblast growth |
| | | factor receptor 2 |
| | | (FGFR2) |
| DYM | rs833497 | |
| (dymeclin) | | |

The exemplary list of genetic markers (e.g., SNPs) indicating an increased susceptibility to schizophrenia included above is not intended to be exhaustive, but is illustrative. Additional genetic markers (not limited to SNPs and not limited to the SNPs listed above in table 1) may also be used. For example, another form of genetic variation known as “runs of homozygosity” (ROH), whereby for relatively long stretches of a subject's genome both chromosomes are identical, has been suggested as a potential indicator of increased risk of schizophrenia.

A test for genetic susceptibility may include a plurality of different markers, including any of those (e.g., all or a subset of those) listed in Table 1, above. For example, the test or screen for genetic susceptibility may include a sub-set of the markers listed above. In some variations, the markers may be ranked or weighted, so that some markers may have a greater indicative power (either alone or in combination with one or more other, or adjunct) markers. For example, markers may be weighted based on the strength of the correlation to schizophrenia (e.g., full-blown DSM schizophrenia).

In some variations the test for genetic susceptibility may look at protein expression rather than just genotype (e.g., proteomics). For example, expression levels of proteins implicated in the brain developmental and neuro-immune pathways, including any of those included above in table 1, may be examined. Protein expression may be examined, for example, by quantitative antibody screening, or any other appropriate methods.

Any indicator reflecting the status of the blood-brain barrier may be used. In particular the markers S100B and MMP-9 may be used as part of an assay (including a serum-based assay). S100B is typically not found at high concentration in the blood, but is found at higher concentrations in brain (e.g., cerebrospinal) fluids. Thus if blood levels of S100B are elevated, the blood-brain barrier may be weakened. Similarly, MMP-9 is an enzyme that is known to weaken the blood-brain barrier when levels become elevated. Thus, if MMP-9 serum levels are elevated, the blood-brain barrier may be weekend. As described herein, the MMP-9 protein may be a therapeutic target for the treatment of prodromal schizophrenia.

Any appropriate indicator of an ongoing (e.g., currently present) pro-inflammatory state may be used as well. For example, markers of active inflammatory process may include, but are not limited to, measurements of complement, TNF alpha, IL-1,6,7,10, IFN gamma, transferrin and haptoglobin via quantitative reverse PCR. In some variations proteins such as soluble TNF-R1 (TNF alpha receptor-1) protein may be assayed, as may levels of S100B, and/or MMP-9 (a non-specific biomarker of increased blood brain barrier permeability). These soluble proteins may provide an indication of inflammation, particularly in the brain.

Table 2, below lists some of the markers and/or tests that may be examined when determining if a subject is undergoing inflammation and/or to determine the status of the blood-brain barrier. For example, matrix metalloproteinases (MMPs) are suggested to play important roles in autoimmune disease, chronic infections and recently in schizophrenia, and may be examined to determine inflammation. The MMP level (e.g., MMP 9) may be examined in comparison with a baseline value, or in comparison with other markers. MMP-9, a member of the matrix metalloproteinase family that degrades collagen IV and processes chemokines and cytokines, participates in response to stress and injury. Up regulation allows leukocytes to travel through lymphatics and may provide an indirect marker of increased blood brain barrier permeability. TNF also induces MMP-9, thus it may be an indirect marker for TNF increase. Levels and activities of plasma MMP-9 can be investigated by enzyme-linked immunosorbent assay and gel zymography. An MMP-9/TIMP-1 ratio can also be calculated. A haptoglobin-MMP-9 Elisa may be a serological means to measure. Normal values of MMP are expected in the range of 40 ng/m, while salivary levels (e.g., levels in saliva) of MMP-9 are typically >20 ngmL, and TIMP-1>64 ngmL, thus inflammation may be apparent when the MMP-9/TIMP-1 ratio is >1, in some variations.

TABLE 2
Markers that may be used to determine an inflammatory
state (including pro-inflammatory markers) associated
with prodromal schizophrenia

| Marker | Comments |
|---|---|
| TNF receptor (TNF alpha) | Can measure from blood or other |
| | bodily fluid by immunoassay |
| IL-1 | Can be measured by immunoassays |
| | (e.g., ELISA CLEA, other enzyme- |
| | linked immunoassay, etc.) |
| IL-6 | Can be measured by immunoassays |
| IL-7 | Can be measured by immunoassays |
| IL-10 | Can be measured by immunoassays |
| IFN gamma | Can be measured by immunoassays |
| transferrin | Can be measured by immunoassays |
| haptoglobin | Can be measured by immunoassays |
| MMP-9 | Can be measured by immunoassays |
| S100B | Can be measured by immunoassays |

Alternatively or additionally, any appropriate indicator of an ongoing (e.g., currently present) indicator of oxidative stress may be used. For example, direct or indirect measures of oxidative stress may be used, such as measurements of cysteinylated or glutathionylated proteins and other thiol compounds via liquid chromatography, mass spectrometry, or redox based isotopes which can measure oxidation of specific cystines. Tests or assays for TBARS (ThioBarbituric Acid Reactive Substances Assay), urinary isoprostanes, etc., may be used.

In one example, an assay that may be used in helping to determine if a subject is prodromal schizophrenic may include a sub-stet of the markers reflecting the status of the blood-brain barrier and inflammation. For example a screen for status of the blood-brain barrier relevant to prodromal schizophrenia may include: TNF, Il-1, IL-6, Haptoglobin, MMP-9, and S100B. In some variations this is a serological panel. In some variations, the panel may be performed in conjunction with a panel or panel examining the markers for genetic susceptibility described above.

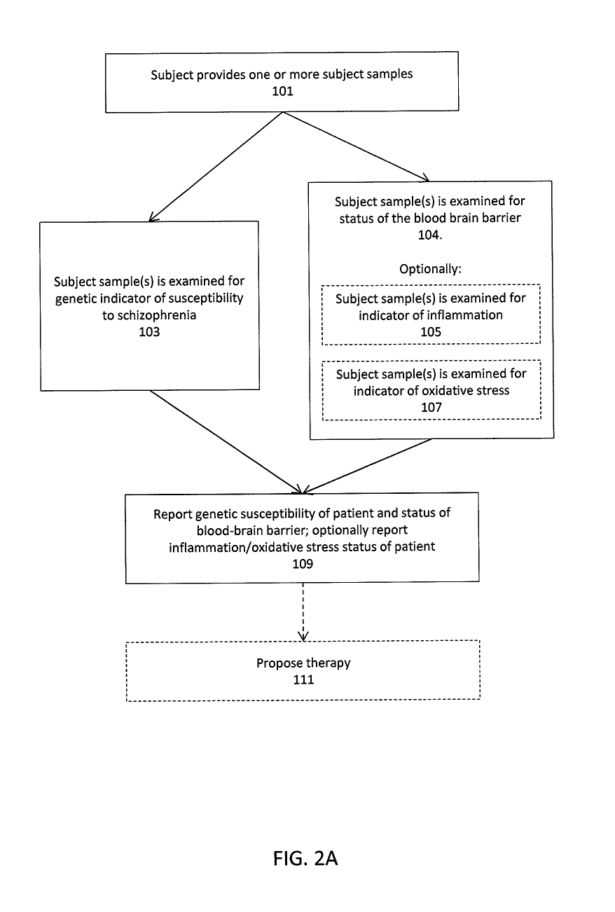

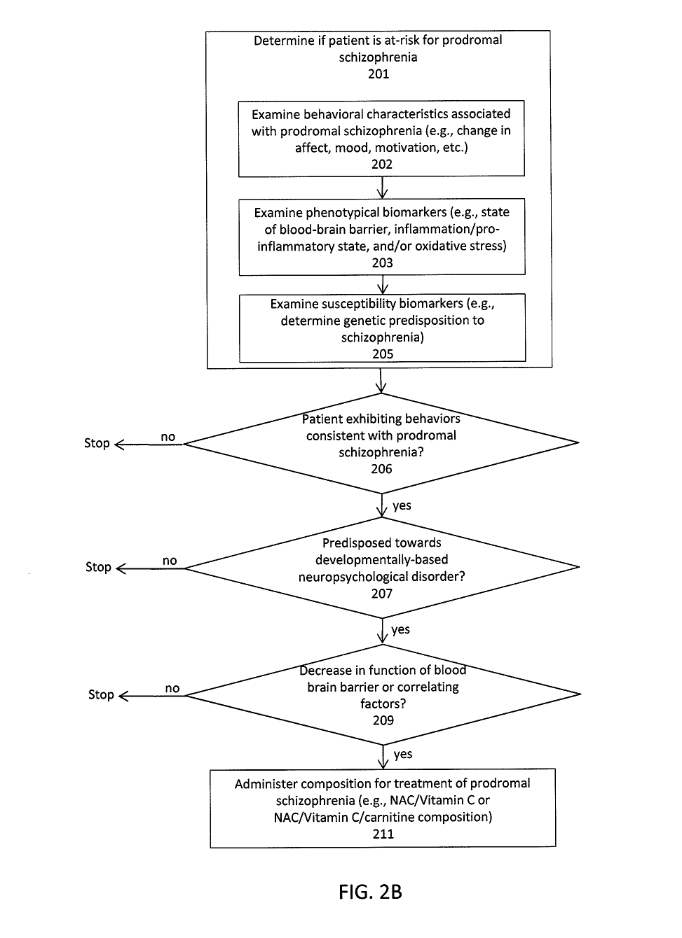

FIG. 2A illustrates one variation of a method of identifying prodromal schizophrenia. The subject may be pre-screened by the physician to determine that he/she is experiencing or exhibiting characteristics consistent with prodromal schizophrenia such as those described above (e.g., neurotic symptoms, mood-related symptoms, changes in volition, cognitive changes, physical changes, behavioral changes and additional symptoms). In FIG. 2A, the subject provides one or more subject samples 101. For example, the subject may provide a single sample (e.g., saliva, blood, tissue, urine, etc.) or multiple samples. These samples may then be examined, either separately or preferably in parallel, for both: (1) one or more indicators of genetic susceptibility for schizophrenia 103; and (2) and one or more indicators of the status of the blood-brain barrier. In some variations, the sample is examined for an ongoing inflammatory state 105 and/or one or more indicators of oxidative stress 107. The oxidative/inflammatory state(s) may be determined in addition or in place of (as proxy for) determining the status of the blood-brain barrier.

Screening the subject for both genetic susceptibility to schizophrenia and the status of the subject's blood-brain barrier (and/or ongoing inflammation/oxidative stress) may be performed as part of a single kit or panel. For example, in some variations the system includes a screen examining one or more genetic risk factors (e.g., all or a subset of the SNPs listed in table 1, above) and a screen for markers indicating status of the blood-brain barrier and/or either or both inflammation (e.g., examining markers or correlates for inflammation, and particularly inflammation of the subject's brain) and/or oxidative stress (e.g., examining markers or correlates for oxidative stress, particularly in the brain). In some variations a panel for confirming prodromal schizophrenia may include only markers indicating the status of the blood-brain barrier and/or inflammation/oxidative stress. For example, when the subject has been predetermined to (1) exhibit behaviors consistent with prodromal schizophrenia and (2) have a susceptibility to schizophrenia (e.g., by personal or family history, or other genetic screen).

A report of the results may be optimized to simplify the risk and treatment of prodromal schizophrenia. The report may aggregate the schizophrenia susceptibility risk with the elements considered to trigger prodromal schizophrenia, such as inflammation and/or oxidative stress and/or the status of the blood-brain barrier. Ultimately, the report may provide an explicit indication of prodromal schizophrenia with one or more metrics (e.g., the likelihood of prodromal schizophrenia, the likelihood of susceptibility to schizophrenia, the presence or degree of inflammation and/or oxidative stress, etc.). In some variations the report may indicate which markers of susceptibility were examined, as well as which indicators of the status of the blood-brain barrier and/or inflammation and/or oxidative stress

Thus, described herein are reports providing an indication of a subject's risk of prodromal schizophrenia. The report may include a calibrated risk level. For example, the risk level may be provided as a percentage (of a 100%), a numeric value (including a unit less score), a qualitative score (e.g., “low, medium, high”), a population ranking (e.g., indicating subject location on a population distribution), or the like. The report may include a breakdown of the susceptibility and the subject's inflammatory state and/or oxidative stress state. As with the subject's prodromal schizophrenia state, any of these sub-elements reported on the report (e.g., susceptibility, inflammatory/pro-inflammatory state, oxidative stress state) may be indicated with reference to a population (e.g., general population, schizophrenic population, prodromal schizophrenic population, etc.), as an absolute or relative ranking (numeric or quantitative, or qualitative), or the like.

In determining the result to be reported on the report, the subject's susceptibility may be combined with the subject's current inflammation/oxidative stress state. Broadly speaking if the subject has one or more markers linked to an increased susceptibility for schizophrenia, and is also currently experiencing an elevation in pro-inflammatory markers (e.g., inflammation) and/or is under oxidative stress, then the subject is likely in a state of prodromal schizophrenia. Specifically, if the subject is exhibiting behaviors consistent with prodromal schizophrenia, has a genetic susceptibility to schizophrenia, and has a weakened blood-brain barrier, the subject is likely prodromal schizophrenic. The sensitivity of the test may be adjusted by adjusting the ranking of susceptibility and/or the level of inflammation and/or oxidative stress. The presence of one or more markers strongly correlated with schizophrenia and/or multiple markers in any way (weakly, strongly, etc.) correlated with schizophrenia may result in a higher likelihood of genetic susceptibility for schizophrenia which may be combined with the likelihood that the subject is experiencing a weakened blood-brain barrier, and/or inflammation and/or oxidative stress.

Diffusion tensor imaging (DTI) may also be used to confirm or assist in determining the presence or likelihood of a prodromal state. For example, the intervention described herein may also include methods of detection of a prodromal state including MRI Imaging modalities, such as DTI. DTI can provide a probability estimate of a prodromal subject's likelihood of developing schizophrenia using nonparametric density estimator. White matter in the brain typically enables functional networks to transmit signals to different regions of the brain through axonal pathways. Diffusion weighted tensor imaging may provide a means to reveal the human brain's connectivity by providing detailed quantitative analysis of white matter in via in vivo measurement of passive diffusion (random displacement) of water molecules. Information derived from diffusion images can be used to infer the structural organization of white matter. The mobility of water molecules is isotropic and its motion is limited by the presence of tissue components such as cell membranes and fibers. When those elements are aligned, the diffusion becomes directionally preferential and thus anisotropic. In the white matter, axons are organized in parallel bundles and water diffuses preferentially in the direction of the axonal fibers. This anisotropic diffusion in the white matter can be captured by diffusion-weighted images, and is represented by a signal decrease due to diffusive motion in the direction of the applied gradient field. In DTI, the local diffusion is related to the strength of water diffusion along fiber orientation. At each image voxel, diffusion is measured along a set of distinct gradients, producing a corresponding signal. This can provide a Gaussian estimate of the fiber orientation but may be inadequate in regions of crossings and branching fibers, which is what is anatomically typical in the human brain. Diffusion tensors do not follow multivariate Gaussian distributions. Thus, a preferred method to overcome the limitations of DTI for use in prodromal schizophrenia is herein disclosed.

This method computes the orientation probability density function (PDF) at each voxel using a Riemannian framework which does not require that the orientation probability density function be represented by any fixed parameterization, such as a spherical harmonic expansion. Instead, a nonparametric representation of the orientation PDFs which is based upon a Riemannian manifold may be applied in this clinical setting. This method may overcome the inherent non linearity of tensors which limits their clinical applicability by incorporating measurements of geodesic distances on the manifold of axonal pathways. The ability to apply such measurements for use in diagnosing prodromal schizophrenia has not previously been used.

In general, the reports described herein may be written, electronic, oral, text messages, or the like. In particular, the reports described herein may also include some analysis or interpretive guide for understanding and acting upon the results.

As mentioned, if a subject is found likely to be experiencing prodromal schizophrenia, the methods described herein may be used to treat the subject. For example, the subject may be prescribed or given a compound for the treatment of prodromal schizophrenia. For example, the subject may be given a compound/composition for the treatment of prodromal schizophrenia that improves the activity of the blood brain barrier (e.g., making the blood-brain barrier less permeability, restoring normal function, etc.). In some variations the compound/composition is an inhibitor of MMP-9. In some variations, the subject may be given a compound including: NAC, ascorbic acid, lithium and essential fatty acids. Any of the compositors described herein may be marked, labeled or packaged specifically and explicitly for use to treat prodromal schizophrenia.

FIG. 2B illustrates one exemplary method of treating a subject, which may include the steps of identifying the subject likely to have prodromal schizophrenia, as described in FIG. 2A, and treating prodromal schizophrenia when properly identified. For example, in FIG. 2B, the first step 201 includes the identification a patient (i.e. subject, and particularly a child or adolescent) at risk for schizophrenia. This may be achieved by (1) examining the behavior of the subject to confirm that he/she is experiencing behaviors consistent with prodromal schizophrenia 202, (2) examining biomarkers as just described that are indicative of blood-brain barrier status 203. For example, endophenotypes may include, for instance, subclinical psychotic symptoms including transient psychosis, disorganization, etc. Biomarkers which reveal blood-brain barrier status may also be, or be used in conjunction with, biomarkers that increase oxidative stress and/or inflammation markers as described in the paragraphs above. The subject is also either directly analyzed for biomarkers indicative of genetic susceptibility to schizophrenia 205 or a family/personal history indicative of a genetic susceptibility for schizophrenia is completed.

Next, in subjects in which the biomarkers indicate both a susceptibility of developing schizophrenia 207 and a dysfunction of the blood brain barrier, (e.g., and/or an elevated state of inflammation and/or oxidative stress 209). A diffusion tensor imaging study may be subsequently employed to assess the integrity of white matter. Improved methods of determining the integrity of axons are disclosed.

Based upon analysis of diagnosis, the subject may be prescribed and/or administered a compound or composition configured to treat prodromal schizophrenia as mentioned earlier.

While the compositions, methods of forming them, and methods for using them, have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the invention.

In particular, it should be readily apparent to those of skill in the art that the methods and compounds for treating prodromal schizophrenia may be used independently of the methods of determining if a subject is positive for (or at risk for) prodromal schizophrenia. The method and compounds for treating prodromal schizophrenia may be used to treat even subjects for whom prodromal schizophrenia has been determined using other methods than those described herein. Further the compounds described herein may be used for other indications (particularly other neurological disorders or psychosis) and are not limited to prodromal schizophrenia.

What is claimed is:

1. A composition labeled for treatment of prodromal schizophrenia, the composition comprising an agent that restores the function of the blood-brain barrier.

2. The composition of claim 1, wherein the agent that restores the function of the blood-brain barrier comprises an inhibitor of MMP-9.

3. The composition of claim 1, wherein the agent that restores the function of the blood-brain barrier comprises one or more of: doxycycline, minocycline, and valproic acid.

4. The composition of claim 1, further comprising an N-acetyl cysteine (NAC) compound.

5. The composition of claim 1, further comprising a lithium compound.

6. The composition of claim 1, further comprising an essential fatty acid.

7. A composition for treating prodromal schizophrenia, the composition comprising:
an N-acetyl cysteine (NAC) compound in a first amount by weight;
a lithium compound;
a fatty acid compound.

8. The composition of claim 7, wherein the concentration of the lithium compound is approximately 1 mg/kg.

9. The composition of claim 7, wherein the fatty acid compound is between about 1 and 0.1 percent of the first amount.

10. The composition of claim 7, wherein the fatty acid compound comprises an essential fatty acid.

11. The composition of claim 7, wherein the composition is compounded as a single dose.

12. A method of determining if a subject is experiencing prodromal schizophrenia, the method comprising:
determining if the subject has a genetic susceptibility to schizophrenia;
determining if the subject's blood brain barrier is compromised; and
indicating a likelihood of prodromal schizophrenia based on the presence of a genetic susceptibility for schizophrenia and the compromised status of the blood brain barrier.

13. The method of claim 12, wherein determining if the subject's blood brain barrier is compromised comprises determining the subject's expression or level of one or more of: TNF, IL-1, IL-6, Haptoglobin, MMP-9, S100B.

14. The method of claim 12, wherein determining if the subject's blood brain barrier is compromised comprises examining a level of MMP-9 from the blood.

15. The method of claim 12, further comprising determining if the subject is experiencing inflammation or is under oxidative stress.

16. A method of determining if a subject is experiencing prodromal schizophrenia, the method comprising:
determining if the subject has a genetic susceptibility to schizophrenia;
determining if the subject's blood brain barrier is compromised; and
reporting if the subject is experiencing prodromal schizophrenia based on the concurrent presence of a genetic susceptibility for schizophrenia and a weakened blood-brain barrier.

17. The method of claim 16, wherein determining if a subject has a polymorphisms in a gene associated with a glutamate receptor, or in gene associated with an enzyme of the oxidative pathways related to glutathione and neuregulin.

18. The method of claim 16, wherein determining if the subject's blood brain barrier is compromised comprises determining the subject's expression or level of one or more of: TNF, IL-1, IL-6, Haptoglobin, MMP-9, S100B.

19. The method of claim 16, wherein determining if the subject's blood brain barrier is compromised comprises examining a level of MMP-9 from the blood.

20. The method of claim 16, further comprising using diffusion tensor imaging to confirm the presence of prodromal schizophrenia.

21. A method of preforming diffusion tensor imaging (DTI) comprising:
taking a magnetic resonance image (MRI) of a subject's brain;
computing the orientation probability density function (PDF) at each voxel of the MRI image using a Riemannian framework that does not require that the orientation probability density function be represented by any fixed parameterization, wherein a nonparametric representation of the orientation PDFs is based upon a Riemannian manifold.

22. A method of treating a subject with prodromal schizophrenia, the method comprising providing a subject experiencing prodromal schizophrenia with a composition to improve, repair, or prevent further damage to the blood-brain barrier.

23. A method of treating a subject with prodromal schizophrenia, the method comprising providing a subject experiencing prodromal schizophrenia with a composition to inhibit MMP-9.

24. The method of claim 20, further comprising determining if the subject is prodromal for schizophrenia by obtaining diffusion tensor imaging to probe the integrity of white matter and to refine such assessments by incorporating geodesic distances based upon a Riemann manifold.
